(19) United States
(12) Patent Application Publication
(10) Pub. No.: US 2016/0359168 A1
(43) Pub. Date: Dec. 8, 2016

(54) HIGH VOLTAGE ORGANIC MATERIALS FOR ENERGY STORAGE APPLICATIONS

(71) Applicants: TOYOTA MOTOR EUROPE NV/SA, Brussels (BE); UNIVERSITA DEGLI STUDI DI MILANO BICOCCA, Milano (IT)

(72) Inventor: Fabio ROSCIANO, Schaarbeek (BE)

(73) Assignee: Toyota Motor Europe NV/SA, Brussels (BE)

(21) Appl. No.: 15/102,946

(22) PCT Filed: Dec. 12, 2013
(86) PCT No.: PCT/EP13/76398

§ 371 Date: Jun. 9, 2016

(51) Int. Cl. H01M 4/60; H01M 10/0525; H01M 4/36; C08G 61/12; C07D 519/00

(52) U.S. Cl. CPC H01M 4/608; C08G 61/124; C07D 519/00; H01M 4/364; H01M 10/0525; C08G 2261/3241; C08G 2261/51; H01M 2004/028

(57) ABSTRACT

The present invention relates to indolizine-based materials as energy storage materials in an electrochemical energy storage device. In preferred embodiments, said indolizine-based materials are obtained by electropolymerization, or chemical polymerization, of small molecule indolizines, in particular ones which have one or two indolizine ring systems.

### BACKGROUND TO THE INVENTION

Rechargeable (secondary) batteries are of increasing importance both in the consumer electronics field (as components of e.g. mobile telephones and laptop computers) as well as in vehicle and aerospace applications. An example of a rechargeable battery is the lithium-ion battery in which lithium ions in the electrolyte move from the negative electrode to the positive electrode during discharge (and move in the reverse direction during charging). FIG. 1 shows an example of an electrochemical device used to store energy (a battery) containing a positive electrode material and a negative electrode material separated by separator containing a liquid, gel, polymeric or solid electrolyte, with a current collector used on both sides of the battery to carry the electrical energy.

Organic materials can be used effectively to store charge. Thus, US 2010/0009256 and US 2008/0038636 relate to the use of a polyradical material, a polymer with pendant nitroxyl radical groups, as an electrode active material. US 2003/0096165 discloses materials containing polyradicals of various structures for use in secondary batteries, and U.S. Pat. No. 7,045,248 focuses on boron or sulfur radicals.

In order to increase the energy density of electrode materials for electrochemical energy storage, two strategies can be applied: (1) increase of the specific capacity Q (Ah/kg or Ah/L), or (2) increase of the electrochemical potential V at which the reaction occurs. The energy density (Wh/kg or Wh/L) is defined as E=Q*V, thus increasing V directly increases the energy density E.

Organic radicals based on the 2,2,6,6-tetramethylpiperidine-1-oxyl group (TEMPO) have been thoroughly investigated as cathode materials in organic batteries. This group shows an oxidation potential close to 3.6 V vs. Li⁺/Li (Electrochimica Acta 2004 50(2-3) 827-831, also EP 1 128 453). Other materials have been used as possible cathode material for batteries, but their potential is lower than the above, such as dilithium rhodizonate (3 V vs Li⁺/Li, ChemSusChem 2008 1 348-355); Tetracyanoquinodimethane (3 V vs. Li⁺/Li, Scientific Reports 2012 2 453); or purpurin (3.2 V vs. Li⁺/Li, Scientific Reports 2012 2 960).

Independently of the above, the use of indolizine in batteries has been described, but as cations of an ionic liquid to be used as electrolyte (EP 2169756, U.S. Pat. No. 6,472,100).

### SUMMARY OF THE INVENTION

It is an object of the present invention to provide electrode materials for electrochemical energy storage, which have a high energy density, notably due to a high electrochemical potential. Molecules described herein comprising the indolizine structure offer an oxidation potential of 3.8 V vs. Li⁺/Li, thus showing about a 10% improvement over the state of the art. The synthetic work of the present inventors has provided materials based on the indolizine structure that can be incorporated in an electrochemical energy storage device such as a battery.

In one aspect, the present invention therefore relates to a battery comprising a positive electrode, a negative electrode and an electrolyte therebetween, wherein either the positive electrode or the negative electrode contains an indolizine-based material. In preferred embodiments, the positive electrode/cathode contains the indolizine-based material.

In a further aspect, the invention relates to the use an indolizine-based material as an energy storage material in an electrochemical energy storage device.

In preferred embodiments, the said indolizine-based materials are obtained by electropolymerization, or chemical polymerization, of small molecule indolizines, in particular ones which have one or two indolizine ring systems. Advantageous indolizine ring substitution patterns for such molecules, that can be appropriately used in the framework of the present invention, will be set out in the detailed description of the invention that follows.

In a further aspect, the invention relates to the following compound:

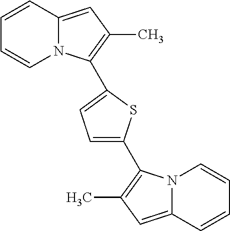 [SMILES: Cc1cc2ccccn2c1-c1ccc(-c2c(C)cc3ccccn23)s1]

which has been prepared in the context of the research of the present invention into new energy storage materials.

### BRIEF DESCRIPTION OF THE FIGURES

FIG. 1 shows an example of an electrochemical device used to store energy (a battery), in a schematic representation.

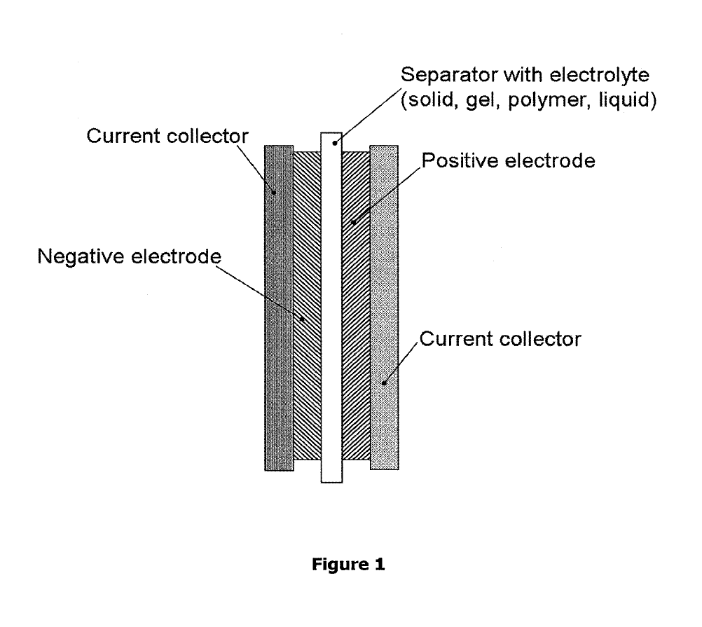

FIG. 2 shows the structures of the five indolizine molecules specifically studied in the present invention.

FIG. 3 is part of the prior art and shows electrochemical activity of the TEMPO organic radical.

FIG. 4 shows electrochemical activity of all of the electropolymerized “poly”-indolizines specifically studied in the present invention.

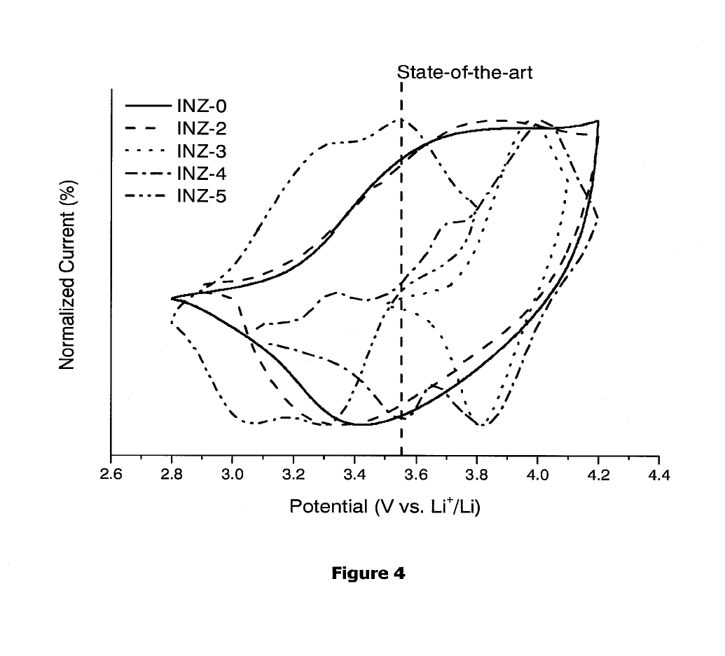

FIG. 5(a-e) shows the electropolymerization of all the indolizine molecules specifically studied in the present invention.

FIG. 6(a-e) shows the electrochemical characterization of all the indolizine-based layers obtained by electropolymerization process.

FIG. 7 shows an example of battery performances obtained using the INZ-5-based layer obtained by electropolymerization as positive electrode material.

FIG. 8 shows an example of a typical charge-discharge curve of a “metal-free” battery obtained using the INZ-4 active material as cathode and the NPbIm-1 active material as anode, and a 1M tetrabutylammonium perchlorate solution in acetonitrile as electrolyte.

### DETAILED DESCRIPTION OF THE INVENTION

The indolizine molecule, without any substitution, has the following structure:

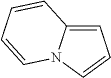

The indolizine core ring system, because of its high potential operation and low molecular weight, may enable a theoretical capacity of 230 mAh/g to be obtained. However, the molecule should appropriately be modified and functionalized in order to be useful for energy storage.

Modifications of the indolizine molecule may inter alia help to increase the operation potential and/or to improve the stability towards electrochemical oxidation and reduction.

The generic structure of the molecules used in this invention can be summarized as shown in the following two schematic diagrams. The first diagram (formula (I)) shows a single indolizine-ring containing molecule that can be used as the basis of polymers having said indolizine as repeating units, according to the present invention:

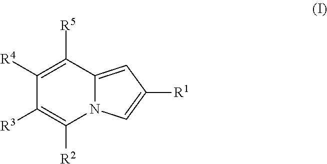

Advantageously, in the present invention, in above formula (I), the 5-membered ring positions are not substituted (bearing only hydrogen (H) atoms), apart from the carbon atom bearing the R¹ group, because these positions on the 5-membered ring are involved in polymerization, important in the preparation of indolizine-based materials for energy storage purposed in the present invention.

The following, second diagram (formula (II)) shows a class of molecules having two indolizine rings connected through a conjugated bridge. Such molecules can also be polymerized to give polymers having said indolizines as repeating units:

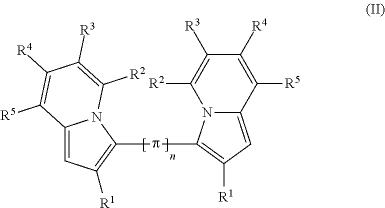

In both structures, R¹ can be a hydrogen atom or any linear or branched alkyl chain, or glycolic chain, having from 1 to 8 carbon atoms, an ester whose alcoholic residue is an alkyl chain which is linear or branched having up to 8 carbons/an ester having up to 10 carbon atoms in all, a benzene ring, or a naphthalene ring substituted at the 1 or 2 position. For example, in advantageous embodiments R¹ may be -Me or —C₆H₅, -Me being particularly preferred. R², R³, R⁴, and R⁵ may be substituents including, without being limited to, the following: H, methyl, branched or linear alkyl chains up to 8 carbon atoms, halogens, esters and amides preferably with 8 or less carbon atoms, alkyl and aryl nitriles preferably with 8 or less carbon atoms, nitro derivatives, sulfones and sulfoxides, perfluoroalkyl preferably with 8 or less carbon atoms, alkoxy groups, dialkylamino, diarylamino, phenyl, 1-naphthyl, 2-naphthyl. The reference t of formula (II) indicates a conjugated bridge, which may be a double bond, a triple bond, or a plurality of double or triple bonds conjugated in a number of 2 or 3, e.g. a benzenic ring, a thiophenic ring, a furane ring and a pyridine ring. Such rings can be functionalized with residues having the same nature as substituents R²-R⁴. In advantageous embodiments, π may comprise a (carbon-carbon) double bond e.g. π may be —CH═CH—, which may be in a trans configuration), or a phenyl ring, for example —C₆H₄—, for example in a para- (or meta-) substitution pattern. Alternatively, π may be a conjugated heterocyclic system such as a 2,5-thienyl bridge. n in formula (II) is an integer of at least 1, and is preferably equal to 1.

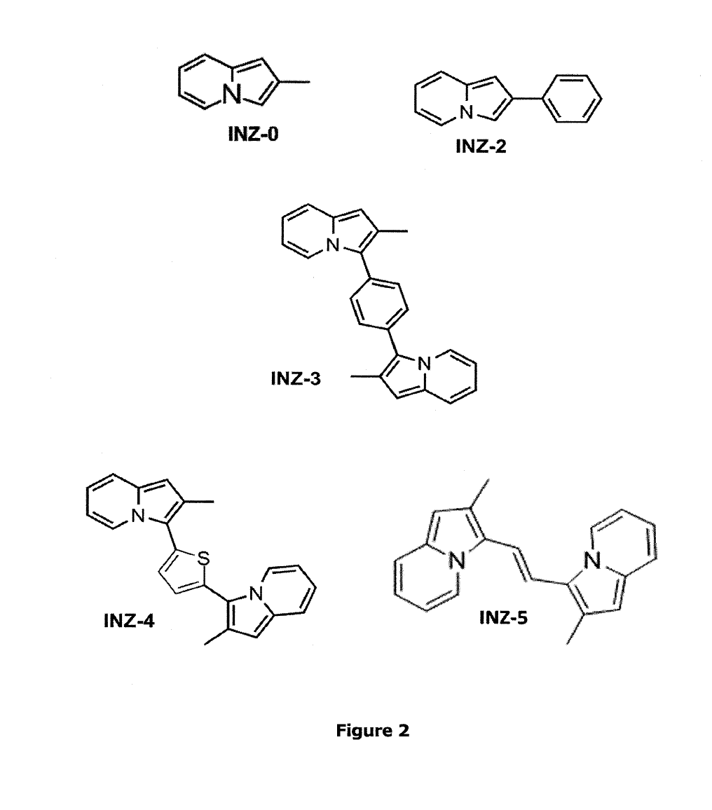

In the experimental work of the present invention, five specific examples of modified indolizines were prepared and the electrochemical behavior of polymers derived therefrom was studied. Synthesis of the materials is contained in part A of the experimental examples section below, while the polymerization protocols are contained in part B. The structures of the five indolizine molecules specifically studied in the present invention are shown in FIG. 2.

In order to prepare indolizines for use as electrochemical energy storage materials, these may be converted into polymers. This can be done according to the standard scheme of oxidative polymerization of electron-rich heterocycles, through the use of oxidizing agents the likes of: Iron(III) salts, cerium ammonium nitrate, iodine, ammonium persulfate and in general any oxidizing agent able to generate the radical cation of the aforementioned indolizine monomers.

In a typical secondary battery, a negative electrode layer and a positive electrode layer are separated via a separator containing an electrolyte. A positive electrode collector may be attached to the positive electrode layer, and a negative electrode collector may be attached to the negative electrode layer.

The negative electrode collector and the positive electrode collector may be a metal foil or metal plate made of, for example, nickel, aluminum, copper, gold, silver, an aluminum alloy and stainless steel; a mesh electrode; and a carbon electrode. The collector may be active as a catalyst or an active material may be chemical bound to a collector. A separator made of a porous film or a nonwoven fabric may be used for preventing the above positive electrode from being in contact with the negative electrode. The separator may, for example, be made of polyethylene or polypropylene, or glass fiber.

The indolizine-based organic active material of the invention is preferably used at the positive electrode. It is possible to combine the indolizine-based organic active material with other, known positive electrode active materials.

Such species may include lithium manganates such as LiMnO₂ and LiₓMn₂O₄ (0<x<2), lithium manganates having a Spinel structure, MnO₂, LiCoO₂, LiNiO₂, Li_{y}V₂O₅ (0<y<2), olivine materials LiFePO₄, and materials in which a part of Mns in Spinel structure are substituted with another transition metal.

As materials for the negative electrode layer, these may appropriately include carbon materials such as graphite and amorphous carbon, lithium metal or a lithium alloy, lithium-ion occluding carbon and conductive polymers. Film, bulk, granulated powder, fiber and flake forms of such materials may be used. Apart from lithium, other metals may be used at the negative electrode, such as sodium and magnesium. It is also possible to use calcium, silver, copper and aluminum as metal anodes.

In advantageous embodiments of the present invention, the positive electrode or the negative electrode (most preferably the positive electrode) contains at least 50% by mass of the indolizine-based material, more preferably at least 60% by mass, still more preferably at least 70% by mass, even more preferably at least 80% by mass, and even in some embodiments at least 90% by mass.

A conductive auxiliary material or ion-conductive auxiliary material may be added for reducing an impedance during forming an electrode layer comprising the indolizine-based organic active material of the invention (normally in the positive electrode), and/or in the opposite electrode. Examples of such a material include carbonaceous particles such as graphite, carbon black and acetylene black and conductive polymers such as polyaniline, polypyrrole, polythiophene, polyacetylene and polyacene.

A binder may be used for reinforcing binding between components in either electrode. Examples of suitable binders include polyvinylidene fluoride, a copolymer of vinylidene fluoride and hexafluoropropylene, a copolymer of vinylidene fluoride and tetrafluoroethylene, polytetrafluoroethylene, a copolymer rubber of styrene and butadiene, and resin binders such as polypropylene, polyethylene and polyimide.

An electrolyte contained in the battery transfers charged carriers between the electrodes, and may be prepared by, for example, dissolving an electrolyte salt in a solvent. Examples of such a solvent include organic solvents such as ethylene carbonate, propylene carbonate, dimethyl carbonate, diethyl carbonate, methyl ethyl carbonate, γ-butyrolactone, tetrahydrofuran, dioxolane, sulforane, dimethylformamide, dimethylacetamide, N-methyl-2-pyrrolidone, acetonitrile. Other possibilities include sulfones (e.g. ethyl-methyl sulfone), sulfoxides (DMSO, dimethylsulfoxide), ethers such as tetraglyme. In this invention, these solvents may be used alone or in combination of two or more. Examples of an electrolyte salt include LiPF₆, LiClO₄, LiBF₄, LiCF₃SO₃, LiN(CF₃SO₂)₂, LiN(C₂F₅SO₂)₂, LiC(CF₃SO₂)₃ and LiC(C₂F₅SO₂)₃. Non-metal electrolyte salts include tetrabutylammonium (TBA) salts such as TBA-ClO₄.

An electrolyte may be solid. Examples of a polymer used in the solid electrolyte include vinylidene fluoride polymers such as polyvinylidene fluoride, a copolymer of vinylidene fluoride and hexafluoropropylene, a copolymer of vinylidene fluoride and ethylene, a copolymer of vinylidene fluoride and monofluoroethylene, a copolymer of vinylidene fluoride and trifluoroethylene, a copolymer of vinylidene fluoride and tetrafluoroethylene and a terpolymer of vinylidene fluoride, hexafluoropropylene and tetrafluoroethylene; acrylonitrile polymers such a copolymer of acrylonitrile and methyl methacrylate, a copolymer of acrylonitrile and methyl acrylate, a copolymer of acrylonitrile and ethyl methacrylate, a copolymer of acrylonitrile and ethyl acrylate, a copolymer of acrylonitrile and methacrylic acid, a copolymer of acrylonitrile and acrylic acid and a copolymer of acrylonitrile and vinyl acetate; polyethylene oxide; a copolymer of ethylene oxide and propylene oxide; and polymers of these acrylates or methacrylates. The polymer may contain an electrolyte solution to form a gel or the polymer may be used alone.

A secondary battery in the present invention may have a conventional configuration, where, for example, an electrode laminate or rolled laminate is sealed in, for example, a metal case, a resin case or a laminate film made of a metal foil such as aluminum foil and a synthetic resin film. It may take a shape of, but not limited to, cylindrical, prismatic, coin or sheet.

A secondary battery according to the present invention may be prepared by a conventional process. For example, a slurry of an active material in a solvent is applied on an electrode laminate and the product is piled with a counter electrode via a separator. Alternatively, the laminate is rolled and placed in a case, which is then filled with an electrolyte solution.

In the present invention, there is disclosed an electrochemical energy storage device (e.g. a battery) where at least one of the electrodes is based on the indolizine (INZ) structure. In particular, the indolizine-based material may appropriately be used at the positive electrode. Depending on the choice of electrolyte, the negative electrode can appropriately be a metal (e.g. Li, Na, Mg), an intercalation material (e.g. graphitic carbon or a ceramic such as Li₄Ti₅O₁₂), a lithium conjugated aromatic carboxylate material (e.g. as described by Walker at al., J. Mater. Chem., 2011, 21, 1615-1620), or another organic material (e.g. Naphthalene Bisimide, NPbIm). The electrolyte can be a liquid, a polymer, a solid or a combination of these. In particular, when using two organic materials as electrodes, the salt dissolved in the electrolytic solution can be organic (i.e. no need for metallic cations such as Li⁺, Na⁺, Mg²⁺), thus allowing one to build a completely metal-free battery.

An embodiment of the invention features the indolizine-based material at the cathode, naphthalene bisimide at the anode and an electrolytic solution such as 1M tetrabutylammonium perchlorate (TBAClO₄) dissolved in acetonitrile (ACN). The battery cathode can be prepared by mixing a powder of an INZ material, a conductive additive such as graphitic carbon, and a polymeric binder such as styrene-butadiene rubber in water, thus obtaining a slurry. The slurry can then be cast on an aluminum foil, and dried. After drying, the electrode can be cut and shaped as desired. An analogous process can be employed to prepare the anode, by using a powder of NPbIm in place of INZ. The dried electrodes may appropriately be placed facing each other, a separator such as glass fiber or polyethylene interposed between them, the whole assembly then being flooded with the electrolytic solution. The battery assembly may appropriately be enclosed in an air-tight environment such as a pouch or a can and sealed.

Upon charging the battery, the cathode material is oxidized and the charge is compensated by the anions of the electrolytic solution; at the same time the anode is reduced and the charge is compensated by the cations of the electrolytic solution. The operation of the battery can be summarized as following:

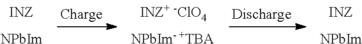

Upon discharge, the compensating ions are released back in the solution, and INZ and NPbIm go back to their original state, thus providing reversible, rechargeable battery performance.

Within the practice of the present invention, it may be envisaged to combine any features or embodiments which have hereinabove been separately set out and indicated to be advantageous, preferable, appropriate or otherwise generally applicable in the practice of the invention. The present description should be considered to include all such combinations of features or embodiments described herein unless such combinations are said herein to be mutually exclusive or are clearly understood in context to be mutually exclusive.

### EXPERIMENTAL SECTION

### Examples

The following experimental section illustrates experimentally the practice of the present invention, but the scope of the invention is not to be considered to be limited to the specific examples that follow.

### Part A: Synthesis of INZ Materials

### 1. Synthesis of INZ-0

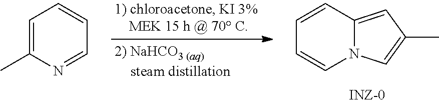

In an amber glass 50 ml RBF, equipped with CaCl₂ guard tube, chloroacetone (10.34 ml, 11.91 g, 128.7 mmol) was added to a solution of 2-picoline (10.00 g, 107.3 mmol) in 2-butanone (25 ml) followed by KI (641 mg, 3.86 mmol). The mixture was heated to 70° C. for 15 h, cooled to RT and kept under stirring for 12 h. Et₂O (25 ml) was added to the mixture and the obtained suspension was filtered on an Hirsh funnel. The deliquescent dark solid was transferred to a 250 ml RBF and dissolved in 125 ml of water. The solution was kept under stirring and NaHCO₃ was slowly added observing gas evolution. The mixture was steam distilled obtaining a suspension of the pure product in the distillate. The distillate was filtered on an Hirsh funnel and the white solid was dried under reduced pressure at room temperature (8.305 g, 63.3 mmol, yield 59%).

¹H NMR δ (ppm, 500.13 MHz, CDCl₃): 7.83 (dd, 1H, j=7.0, j=0.8); 7.31 (d, 1H, j=9.0); 7.14 (s, 1H); 6.66-6.62 (m, 1H); 6.43-6.40 (m, 1H); 6.30 (s, 1H); 2.40 (s, 3H).

### 2. Synthesis of INZ-2

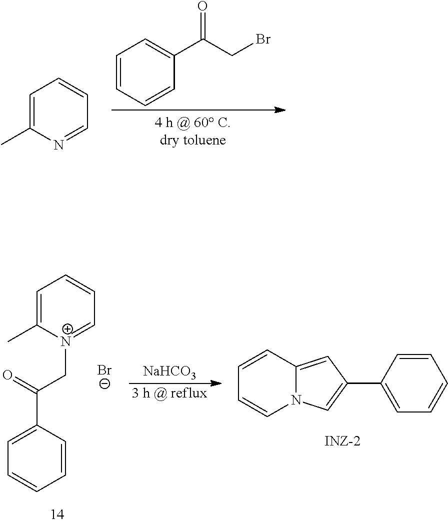

### 2.1 Synthesis of 14

In a 250 ml RBF, equipped with reflux condenser and CaCl₂ guard tube, bromoacetophenone (3.52 g, 37.8 mmol) was added portion wise to a solution of 2-picoline (7.52 g, 37.8 mmol) in dry toluene (90 ml). The obtained solution was stirred at 60° C. for 4 h. Pure product was obtained as a white precipitate that was collected by filtration (10.22 g, 34.98 mmol, yield 92.5%).

### 2.2 Synthesis of INZ-2

Product 14 (10.22 g, 34.98 mmol) was refluxed for 3 h in a solution of NaHCO₃ (3.04 g, 36.19 mmol) in water (150 ml). Product was obtained as a grey precipitate, collected by filtration, and dried under reduced pressure (m.p. 211° C.).

¹H NMR δ (ppm, 500.13 MHz, CDCl₃): 7.89 (d, 1H, j=6.9); 7.67 (d, 2H, j=7.6); 7.58 (s, 1H); 7.40 (t, 2H, j=7.6); 7.35 (d, 1H, j=9.0); 7.26 (t, 1H, j=7.4) 6.70 (s, 1H); 6.66 (t, 1H, j=6.8); 6.46 (t, 1H, j=6.7)

### 3. Synthesis of INZ-3

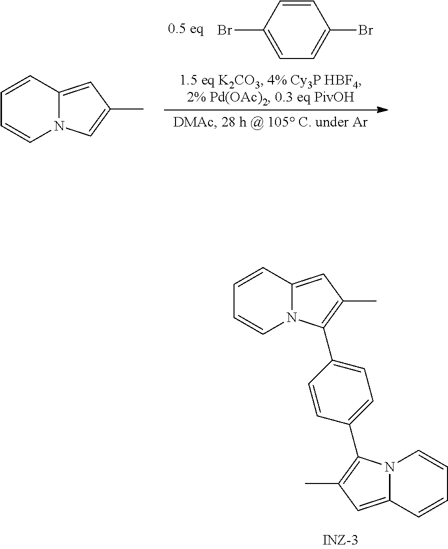

In a screw-capped glass pressure cylinder (total volume 120 ml) a mixture of K₂CO₃ (3.160 g, 22.870 mmol), 1,4-dibromobenzene (1.798 g, 7.624 mmol), Cy₃P HBF₄ (224 mg, 0.610 mmol), Pd(OAc)₂ (68.4 mg, 0.305 mmol) and pivalic acid (467 mg, 4.57 mmol) is prepared in air. The tube is transferred to an Ar-filled glove-box and 2-methylindolizine (2.000 g, 15.24 mmol) is added to the mixture followed by 50 ml of anhydrous DMAc. The tube is tightly closed with a Teflon cap and heated to 105° C. in a heating bath for 28 h. The mixture is allowed to cool to RT, poured in water (200 ml) and extracted with AcOEt (150 ml). The organic phase is collected, washed with brine, dried over MgSO₄ and evaporated under reduced pressure. The crude product is purified by column chromatography (eluent: toluene/n-hexane 1:1). Product is obtained as light grey powder (735 mg, 2.185 mmol, yield 28%).

¹H NMR (500 MHz, CD₂Cl₂) δ [ppm]: 8.19 (d, J=7.1 Hz, 2H), 7.64 (s, 4H), 7.38 (d, J=9.0 Hz, 2H), 6.71 (m, 2H), 6.58-6.33 (m, 4H), 2.43 (s, 6H)

¹³C NMR (125.7 MHz, CD₂Cl₂) δ [ppm]: 133.42, 131.35, 130.84, 124.34, 123.07, 123.009, 119.25, 117.84, 110.61, 101.85, 13.14.

### 4. Synthesis of INZ-4

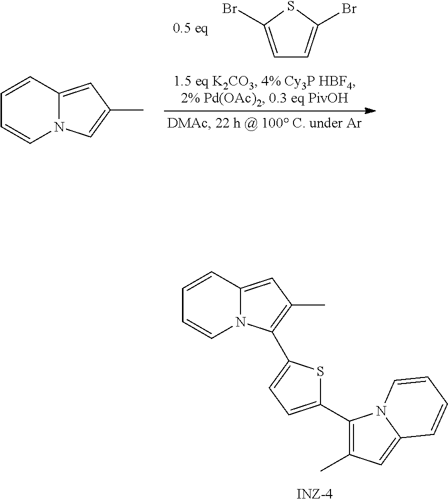

In a screw capped glass pressure cylinder (total volume 120 ml) a mixture of K₂CO₃ (1.580 g, 11.43 mmol), Cy₃P HBF4 (112 mg, 0.305 mmol), Pd(OAc)₂ (34.2 mg, 0.52 mmol) and pivalic acid (234 mg, 2.29 mmol) is prepared in air. The tube is transferred to an Ar-filled glove-box, 2-methylindolizine (1.000 g, 7.62 mmol) and 2,5-dibromothiophene (922.2 g, 3.81 mmol) are added to the mixture followed by 15 ml of anhydrous DMAc. The tube is tightly closed with a Teflon cap and heated to 100° C. in an heating bath for 22 h. The mixture is allowed to cool to RT and filtered, washing with DMAc. The filtrate is poured into water (100 ml) and extracted with CH₂Cl₂ (100 ml). The organic phase is 15 collected, washed with brine, dried over Na₂SO₄ and evaporated under reduced pressure. The crude product is purified by dry flash column chromatography (toluene/n-hexane 1:1) followed by column chromatography (eluent: toluene/n-hexane 1:9->toluene/n-hexane 1:1). Product (684 mg) is crystallized from MeCN under N₂ (566 mg, 1.65 mmol, yield 43%, m.p.: 133.5-134.2° C.).

¹H NMR (500 MHz, CD₂Cl₂) δ [ppm]: 8.34 (d, J=7.0 Hz, 2H), 7.37 (d, J=8.9 Hz, 2H), 7.30 (s, 2H), 6.75 (t, J=7.9 Hz, 2H), 6.55 (t, J=6.8 Hz, 2H), 6.28 (bs, 2H), 2.47 (s, 6H)

¹³C NMR (125.7 MHz, CD₂Cl₂) δ [ppm]: 134.04, 133.67, 128.29, 126.28, 123.69, 119.10, 118.38, 116.12, 111.01, 101.94, 13.44.

### 5. Synthesis of INZ-5

INZ-5 was prepared in two steps through the formation of the 2-methylindolizine-3-carbothialdehyde intermediate:

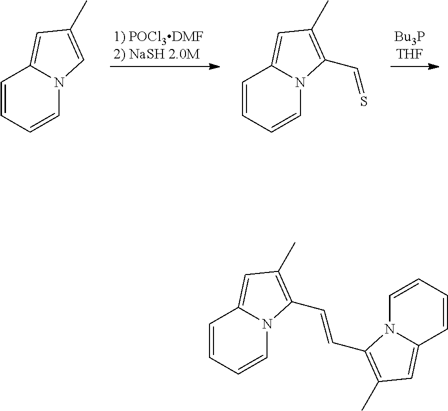

### 5.1 Synthesis of 2-Methylindolizine-3-Carbothialdehyde

In a three-necked flask under N₂ atmosphere, a solution of POCl₃ (1.645 g, 25.15 mmol) in anhydrous DMF (20 ml) was dropped over a period of 30 minutes to a solution of 2-methylindolizine (3.000 g, 22.87 mmol) in anhydrous DMF (20 ml) previously cooled to −50° C. The reaction was kept at this temperature for 1 h, then allowed to reach room temperature. The reaction mixture was then poured into a 2 M solution of NaSH in water (150 ml). A red precipitate was formed and filtered off. The raw solid was purified by column chromatography (alumina, eluent: 9:1 hexane:toluene) giving 2-methylindolizine-3-carbothialdehyde as a brown solid (2.627 g, 66%).

¹H NMR δ (CH₂Cl₂): 11.35 (d, 1H, J=6.8), 10.62 (s, 1H), 7.60 (d, 1H, J=8.5), 7.53-7.50 (m, 1H), 7.13-7.10 (m, 1H), 6.53 (s, 1H), 2.55 (s, 3H)

### 5.2 Synthesis of E-1,2-Bis(2-Methylindolizin-3-Yl)Ethene (INZ-5)

A solution of 2-methylindolizine-3-carbothialdehyde (1.388 g, 7.92 mmol) in anhydrous THF (50 ml) was stirred at r.t. and after the air was replaced by N₂ gas, tributylphosphine (5.768 g, 28.51 mmol) was added. The reaction was kept at reflux temperature till the reagent disappeared (monitored by TLC). The solvent was removed in vacuo, the excess of tributhylphosphine was distilled and E-1,2-bis(2-methylindolizin-3-yl)ethene was purified by filtration on silica gel (eluent: hexane:AcOEt 200:8), yellow powder, yield 28%.

¹HNMR δ (C₆D₆): 7.68 (d, 2H, J=7.3), 7.18 (s, 2H), 6.86 (s, 2H), 6.44-6.40 (m, 4H), 6.17-6.14 (m, 2H), 2.44 (s, 6H)

¹³C NMR δ (C₆D₆): 133.63, 124.97, 122.84, 122.10, 119.34, 117.45, 115.61, 111.08, 103.70, 14.76

### 6. Synthesis of INZ-6

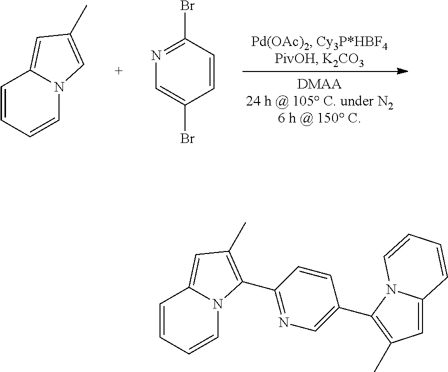

In a screw-capped glass pressure cylinder (total volume 120 ml) a mixture of K₂CO₃ (3.162 g, 22.88 mmol), 2,5-dibromopyridine (1.719 g, 7.26 mmol), Cy₃P*HBF₄ (213.6 mg, 0.58 mmol), Pd(OAc)₂ (65.19 mg, 0.29 mmol) and pivalic acid (445.2 mg, 4.36 mmol) is prepared in air. The tube is transferred in a N₂-filled glove-box and 2-methylindolizine (2.000 g, 15.25 mmol) is added to the mixture followed by 15 ml of anhydrous DMAc. The tube is tightly closed with a Teflon cap and heated to 105° C. in an heating bath for 24 h, then the temperature was raised till 150° C. for 6 h. The mixture is then allowed to cool to RT, poured in water (150 ml) and extracted with AcOEt (150 ml). The organic phase is collected and dried over MgSO₄ and evaporated under reduced pressure. The product has been purified by filtration on silica gel (eluent: hexane gradient hexane:AcOEt 4:1). (yellow powder, 600 mg, yield 24%)

¹H NMR δ (C₆D₆): 9.52 (s, 1H), 8.52 (s, 1H), 7.73 (d, 1H, J=7.1), 7.31 (dd, 1H, J=8.3, J=2.33), 7.26 (d, 1H, J=8.8), 7.18 (d, 2H, J=8.9), 6.54-6.51 (m, 1H), 6.44-6.39 (m, 3H), 6.31-6.28 (m, 1H), 6.07-6.05 (m, 1H), 2.45 (s, 3H), 2.45 (s, 3H)

¹³CNMR δ (C₆D₆): 151.61, 150.47, 137.19, 135.18, 133.99, 126.91, 126.66, 124.93, 124.37, 123.15, 122.62, 121.70, 120.23, 119.70, 119.51, 118.80, 117.99, 111.02, 110.79, 104.02, 102.55, 15.35, 13.07.

### Part B: Polymerization of INZ Materials

### 1. Chemical Polymerization of INZ-0

Under N₂ atmosphere, N-methyl-imidazole (1.313 g, 16.00 mmol) and Baytron-CB-40 (21.7 g, 15.2 mmol) were added to a stirred solution of 2-methylindolizine (INZ-0, 1.000 g, 7.62 mmol) in dry MeCN (40 ml). The mixture was heated to reflux for 7.5 h, cooled to RT and kept under stirring for 3 days. The precipitate was collected by filtration as a dark solid. Product was sonicated and filtered twice with MeCN. The same procedure was repeated with MeOH (2×50 ml). The product was finally filtered and washed with fresh MeOH followed by Et₂O. Residual solvent was removed under reduced pressure at 40° C. Dark solid (1.160 g).

### 2. Chemical Polymerization of INZ-2

Under N₂ atmosphere, N-methyl-imidazole (1.428 g, 17.40 mmol) and Baytron-CB-40 (21.64 g, 15.20 mmol) were added to a stirred solution of 2-phenylindolizine (INZ-2, 1.604 g, 8.30 mmol) in dry MeCN (50 ml). The mixture was heated to reflux for 36 h, cooled to RT and kept under stirring for 1 day. The precipitate was collected by filtration as a dark solid. Product was sonicated with MeCN (40 ml) and filtered. The solid was suspended again in MeCN, stirred for 2 h and filtered. The obtained product was continuously extracted with MeOH in a Soxhlet apparatus for 24 h. Residual solvent was removed under reduced pressure at 65° C. obtaining product as a brown solid (1.425 g).

### 3. Chemical Polymerization of INZ-3

In a dry 2-necked flask, under N2 atmosphere, a mixture of Fe(OTs)₃ (3.944 g, 6.93 mmol), N-methylimidazole and INZ-3 (1.000 g, 2.972 mmol) in anhydrous benzonitrile (10 ml) is heated to 145° C. for 6 h and stirred at room temperature overnight. The mixture is diluted with MeCN, poured in a cellulose thimble and extracted in a Soxhlet extractor (MeOH (4 h), MeCN (8 h), CH₂Cl₂ (8 h)). The residue is dried at 50° C. under reduced pressure for 8 h (737 mg).

### 4. Chemical Polymerization of INZ-4

In a dry 2-necked flask, under N₂ atmosphere, a mixture of Fe(OTs)₃ (9.697 g, 17.03 mmol), and bis(2-methylindolizino)thienylene (2.500 g, 7.30 mmol) in anhydrous benzonitrile (25 ml) is heated to 145° C. for 6 h and stirred at room temperature overnight. The mixture is diluted with MeCN, poured in a cellulose thimble and extracted in a Soxhlet extractor (MeOH (10 h), MeCN (8 h), CH₂Cl₂ (4 h)). The residue is dried at 60° C. under reduced pressure for 8 h (900 mg).

### 5. Electrochemical Polymerization Protocol

Electrochemical polymerization was carried out by cyclic voltammetry. The monomer was dissolved (5 mM) in an electrolyte solution made by 0.1 M TBAClO₄ in ACN. 15 CV cycles between −0.2 and 1.0 V vs. RE were performed in a three-electrode two-compartment cell assembled using:

a 1 cm² (each face) carbon cloth working electrode

a Pt mesh counter electrode (in the separated compartment)

a Ag/AgCl wire as RE (+3.1 V vs. Li).

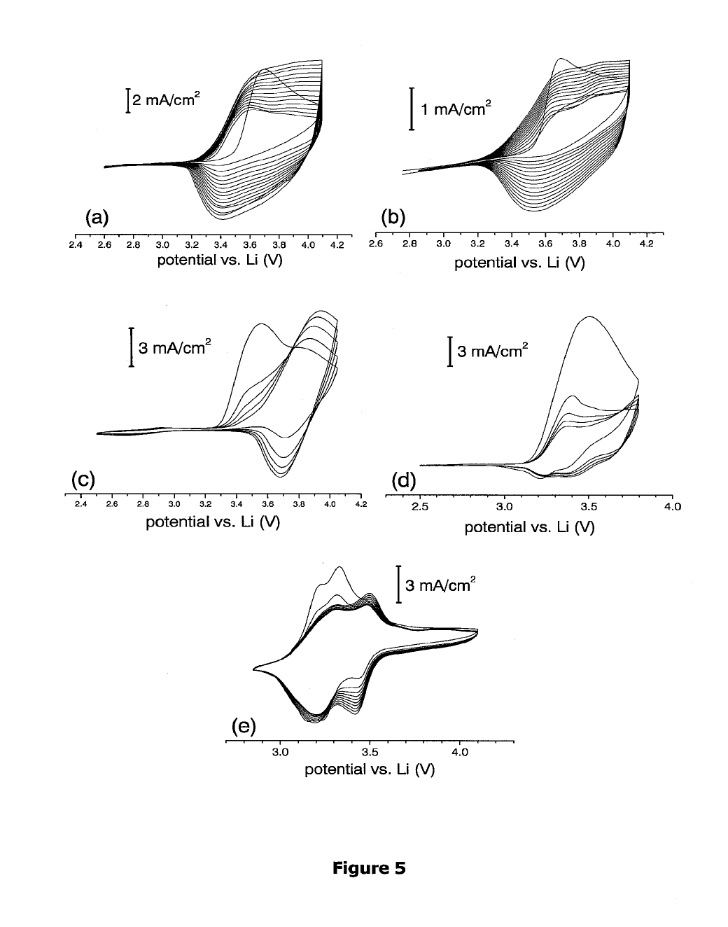

Examples of electrochemical polymerization process for INZ-0, INZ-2, INZ-3, INZ-4, and INZ-5 are reported in FIG. 5 (a), (b), (c), (d), and (e), respectively.

### Part C: Electrochemical Characterization

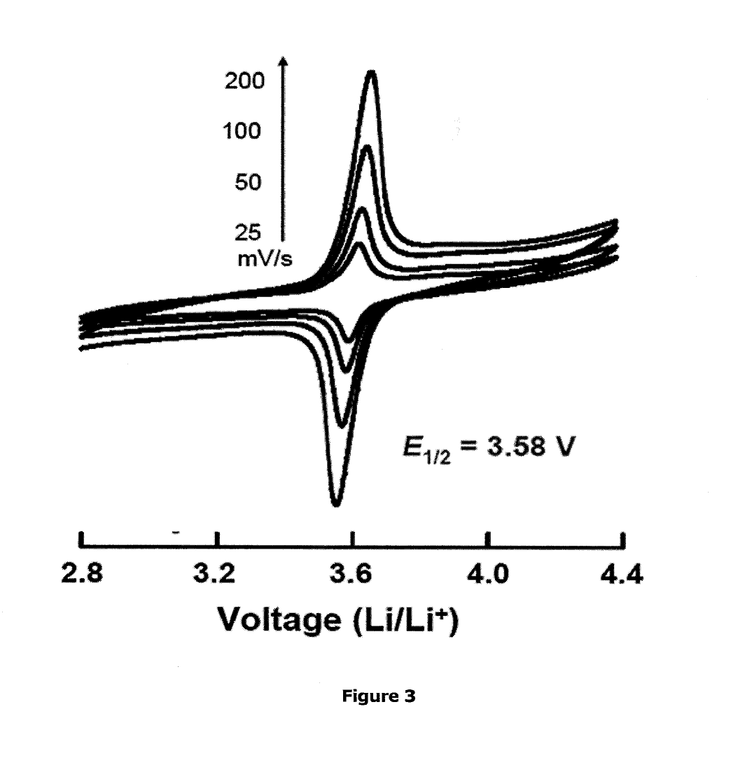

As a state-of-the-art performance of an organic species known for its use as an energy storage materials at a cathode, reference may be made to the performance of the TEMPO organic radical—see in this respect FIG. 3 (adapted from ECS Interface, Winter 2005, p 34). As shown, this state-of-the-art material has electrochemical activity close to 3.5 V vs. Li⁺/Li.

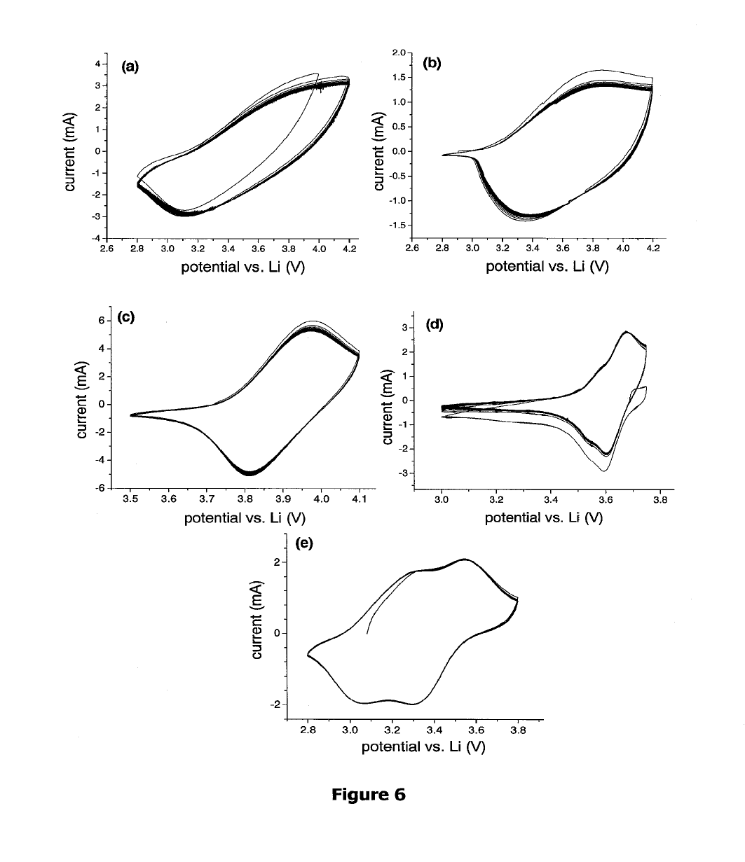

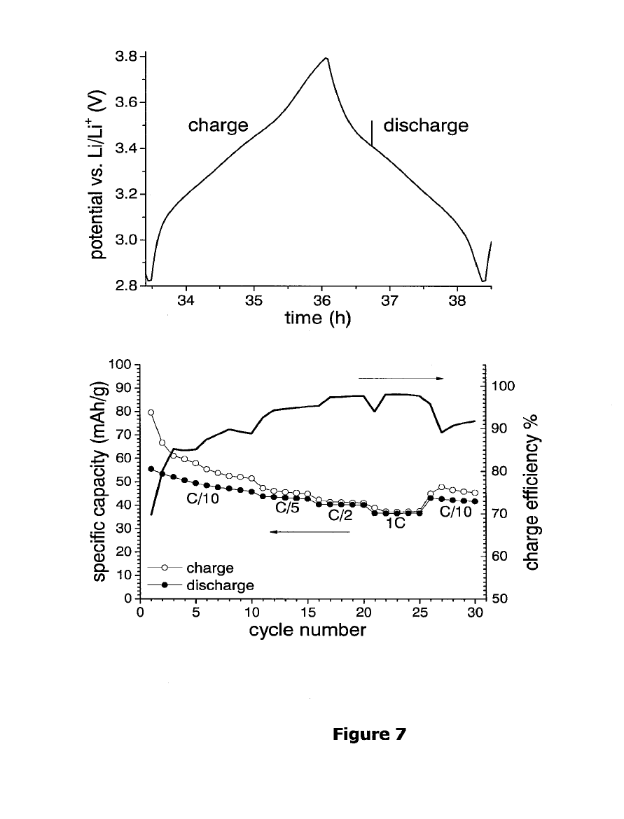

In the present invention, the synthesized molecules INZ-0, INZ-2, INZ-3, INZ-4 and INZ-5 were electropolymerized (FIG. 5) and the resulting layers tested for their electrochemical activity. Cyclic Voltammetries (CVs) were performed in monomer free, 0.1 LiClO₄ propylene carbonate solution at 50 mV/s in flooded cell using metallic lithium foils as both reference and counter electrodes. The resulting current potential profiles for layers obtained from INZ-0, INZ-2, INZ-3, INZ-4, and INZ-5 are reported in FIG. 6 (a), (b), (c), (d), and (e), respectively. An example of battery performances obtained using the layer obtained by INZ-5 and metallic lithium as positive and negative electrodes is reported in FIG. 7. The battery was assembled using a 1 M LiPF₆ EC/DMC commercial electrolyte in a Swagelock cell.

As shown by the data shown in the Figures, it is clear that the indolizine electrochemical activity occurs above 3.8V vs. Li⁺/Li. This represents a 10% increase over the state-of-the-art.

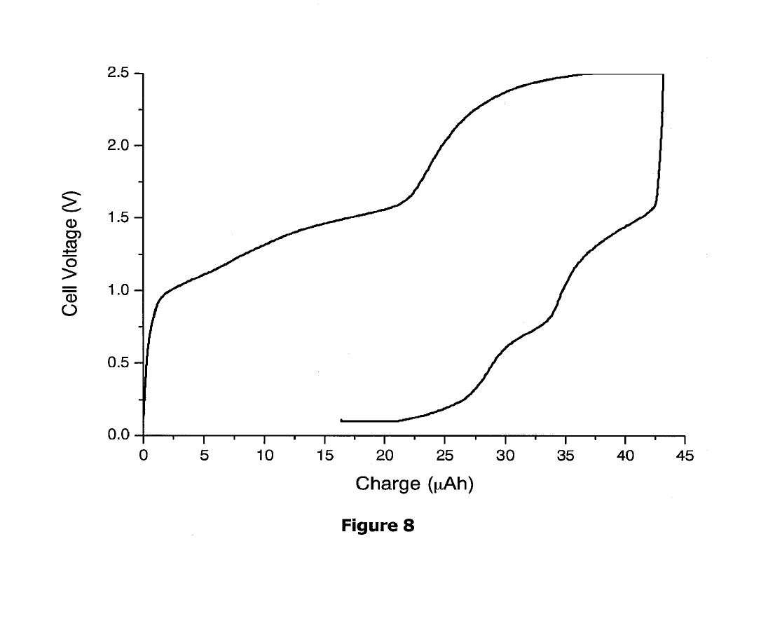

“Metal-free” batteries were prepared as follows: To prepare the positive electrode, INZ-4 was mixed with carbon (Super P) and binder (styrene-butadiene rubber and carboxymethyl cellulose) in a 60:30:10 wt % proportion. Water was added to the mixture until a homogeneous slurry was obtained. Similarly, to prepare the negative electrode, NPbIm-1 was mixed with carbon (Super P) and binder (styrene-butadiene rubber and carboxymethyl cellulose) in a 75:15:10 wt % proportion. Water was added to the mixture until a homogeneous slurry was obtained. Both slurries were then cast on aluminum foil with the Doctor Blade method. The as-cast electrodes were then dried at 80° C. in air overnight. Test electrodes were cut from the dried sheet in the form of circles with a diameter of 16 mm. A “metal-free” electrolyte was prepared as follows: tetrabutylammonium perchlorate was added to acetonitrile to form a 1M solution. The solution is stable at room temperature. Coin-cell type batteries were assembled from the prepared parts. A negative electrode sample was placed as anode material, covered with a glass fiber separator, and the separator was flooded with 250 μl of the electrolyte solution. A positive electrode was placed on top of the separator, and the cell was sealed by crimping. The as-prepared coin cell was cycled in a constant-current constant-voltage pattern (CCCV) using a current of 10 μA/mg in the potential interval 0.1V-2.5V. The cells were placed in a temperature chamber and kept at 25° C. throughout the experiment. A typical charge-discharge curve for the “metal-free” battery is shown in FIG. 8.

1.-10. (canceled)

11. A battery comprising a positive electrode, a negative electrode and an electrolyte therebetween, wherein either the positive electrode or the negative electrode contains an indolizine-based material, wherein the indolizine-based material contains one of the following compounds and/or is prepared by polymerization of one or more compounds according to formula (I) and/or (II) below: 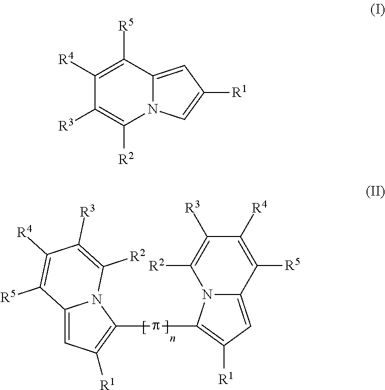
wherein:
R¹ is a hydrogen atom or any linear or branched alkyl chain, or glycolic chain, having from 1 to 8 carbon atoms, an ester whose alcoholic residue is an alkyl chain which is linear or branched having up to 8 carbons/an ester having up to 10 carbon atoms in all, a benzene ring, or a naphthalene ring substituted at the 1 or 2 position;
R², R³, R⁴, and R⁵ are selected from the group consisting of H, methyl, a branched alkyl chain having up to 8 carbon atoms, a linear alkyl chain having up to 8 carbon atoms, a halogen, an ester, an amide, an alkyl nitrile, an aryl nitrile, a nitro derivative, a sulfone, a sulfoxide, a perfluoroalkyl, a alkoxy group, a dialkylamino, a diarylamino, phenyl, 1-naphthyl, and 2-naphthyl;
π in formula (II) may be a double bond, a triple bond, or a plurality of double or triple bonds conjugated in a number of 2 or 3, a benzenic ring, a thiophenic ring, a furane ring or a pyridine ring, such rings being optionally substituted by substituents as defined for R² to R⁵, and n in formula (II) is an integer of at least 1.

12. The battery according to claim 11, wherein the positive electrode contains an indolizine-based material.

13. The battery according to claim 11, wherein the positive electrode or the negative electrode contains at least 50% by mass of the indolizine-based material.

14. The battery according to claim 11, wherein R², R³, R⁴, and R⁵ are selected from the group consisting of an ester with 8 or less carbon atoms, an amide with 8 or less carbon atoms, an alkyl with 8 or less carbon atoms, an aryl nitrile with 8 or less carbon atoms, a perfluoroalkyl with 8 or less carbon atoms, an alkoxy group with 8 or less carbon atoms, and/or a dialkylamino with 8 or less carbon atoms.

15. The battery according to claim 11, wherein n in formula (II) is equal to 1.

16. The battery according to claim 11, wherein a compound of formula (I) or (II) is used wherein R¹ is Me or C₆H₅.

17. The battery according to claim 16, wherein a compound of formula (I) or (II) is used wherein R¹ is Me.

18. The battery according to claim 11, wherein a compound of formula (II) is used wherein π is —CH═CH—, —C₆H₄—, or 2,5-thienyl.

19. The battery according to claim 11, wherein the indolizine-based material is prepared by polymerization of one or more of the compounds having the following structures:

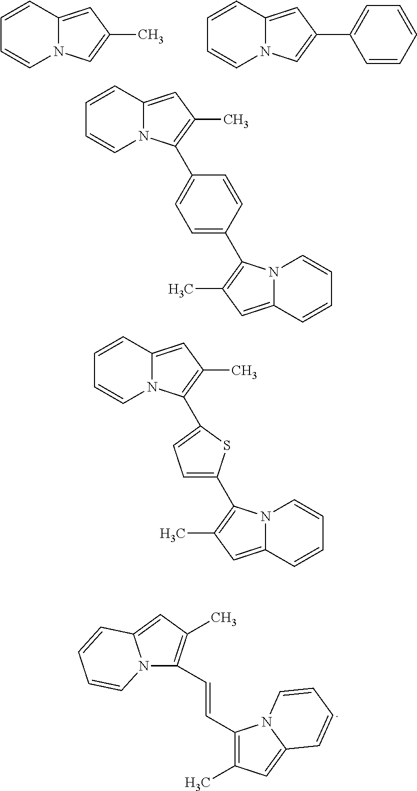

20. An electrochemical energy storage device composing an indolizine-based material, wherein the indolizine-based material contains and/or is prepared by polymerization of one or more compounds according to formula (I) and/or (II) below: 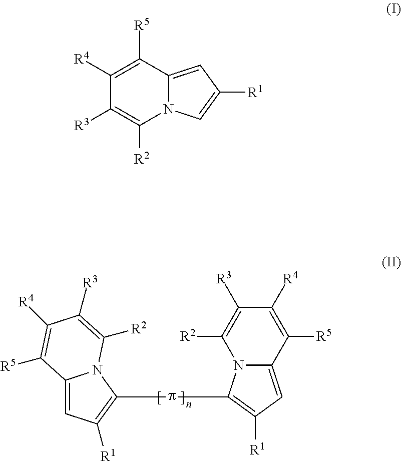
wherein:
R¹ is a hydrogen atom or any linear or branched alkyl chain, or glycolic chain, having from 1 to 8 carbon atoms, an ester whose alcoholic residue is an alkyl chain which is linear or branched having up to 8 carbons/an ester having up to 10 carbon atoms in all, a benzene ring, or a naphthalene ring substituted at the 1 or 2 position;
R², R³, R⁴, and R⁵ are selected from the group consisting of H, methyl, a branched alkyl chain having up to 8 carbon atoms, a linear alkyl chain having up to 8 carbon atoms, a halogen, an ester, an amide, an alkyl nitrile, an aryl nitrile, a nitro derivative, a sulfone, a sulfoxide, a perfluoroalkyl, a alkoxy group, a dialkylamino, a diarylamino, phenyl, 1-naphthyl, and 2-naphthyl;
π in formula (II) may be a double bond, a triple bond, or a plurality of double or triple bonds conjugated in a number of 2 or 3, a benzenic ring, a thiophenic ring, a furane ring or a pyridine ring, such rings being optionally substituted by substituents as defined for R² to R⁵, and n in formula (II) is an integer of at least 1

21. A compound having the following formula: 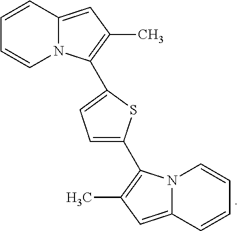 [SMILES: Cc1cc2ccccn2c1-c1ccc(-c2c(C)cc3ccccn23)s1]
